(19) United States
(12) Patent Application Publication
(10) Pub. No.: US 2005/0215904 A1
(43) Pub. Date: Sep. 29, 2005

(54) Ultrasound breathing waveform detection system and method

(75) Inventors: Thilaka S. Sumanaweera, Los Altos, CA (US); Edward A. Gardner, San Jose, CA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/806,875

(22) Filed: Mar. 23, 2004

(51) Int. Cl. A61B 8/02; A61B 8/06; A61B 8/12; A61B 8/14

(52) U.S. Cl. 600/458

(57) ABSTRACT

Cycle information is detected with ultrasound. For example, a breathing cycle is detected from frames of ultrasound data acquired over time. Motion relative to a reference frame is tracked in other frames of data. The variation in motion identifies the cycle. The cycle is then displayed for diagnostic purposes, triggers acquisition of ultrasound images, used for altering other processes or calculations or used for performing another act.

### BACKGROUND

The present invention relates to determining a breathing cycle. In particular, breathing cycle information is used with diagnostic ultrasound imaging.

The breathing cycle of a patient may include diagnostic indicators. The breathing cycle may also be used to trigger ultrasound imaging. For example, contrast agents are injected into a patient and imaged. As another example, a region of interest free of contrast agents is imaged. Quantifications associated with the breathing cycle are calculated from the image data based on the breathing cycle. The imaging may be triggered or synchronized with the breathing cycle. However, the patient typically wears an intrusive and uncomfortable breathing sensor to determine a patient's breathing cycle.

The breathing cycle may alter imaging and quantification. To measure perfusion in an organ, such as the liver or kidney, with ultrasound, added contrast agents or microspheres are injected into a patient. Ultrasound is then used to image the contrast agents as the contrast agents perfuse throughout the organ or tissue of interest. The wash-in or wash-out of contrast agent from the tissue of interest over time is analyzed to determine a rate or amount of perfusion. The time-intensity curve may be inaccurate due to movement. The tissue of interest may move relative to the transducer due to breathing, the effects of the cardiac cycle, unintentional movement of the transducer by the user, or other sources of movement. As a result, the imaged tissue appears to move around within a sequence of ultrasound images. Parameterizing or calculating a time-intensity curve is difficult or inaccurate since a given spatial location in an image may correspond to different locations within the imaged tissue throughout the sequence of images. Due to the breathing or other uncontrollable motion, evaluation of changes that occur at a particular location in an organ or other tissue over time may be erroneous. Triggering based on the breathing cycle may avoid some errors.

Another approach disclosed in U.S. Pat. No. 6,659,953 is to morph images to a reference image, removing the effects of motion. However, tracking errors may accumulate.

### BRIEF SUMMARY

By way of introduction, the preferred embodiments described below include methods and systems for detecting cycle information with ultrasound. For example, a breathing cycle is detected from frames of ultrasound data acquired over time. Motion relative to a reference frame is tracked in other frames of data. The variation in motion identifies the cycle. The cycle is displayed for diagnostic purposes, used to trigger acquisition of ultrasound images, used to alter other processes or calculations or used for performing another act.

In a first aspect, a method is provided for detecting breathing cycle information with ultrasound. Ultrasound data is acquired over a period of time. At least a first portion of a breathing cycle is determined as a function of the ultrasound data.

In a second aspect, a system is provided for detecting breathing cycle information with ultrasound. A memory is operable to store frames of ultrasound data acquired over a period of time. A processor is operable to determine at least a first portion of a breathing cycle as a function of the ultrasound data.

In a third aspect, a method for detecting a cycle with ultrasound data is provided. Motion from a plurality of frames of ultrasound data is tracked with respect to a reference frame of ultrasound data. A cyclic parameter is calculated as a function of the tracked motion. A first portion of the cycle is identified as a function of the cyclic parameter. The tracking, calculating and identifying are repeated for each of a plurality of subsequent cycles. The reference frame of data is reset for each of the subsequent cycles. The reference frame of data is reset to a frame of ultrasound data corresponding to the identified portion of the cycle, such as a minimum or maximum of the cycle.

The current invention is defined by the following claims, and nothing in this section should be taken as a limitation on those claims. Further aspects and advantages are discussed below in conjunction with the preferred embodiments, and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system for morphing ultrasound images;

FIG. 2 is a flow chart diagram of one embodiment of a method for detecting a cycle from ultrasound data;

FIG. 3 is a graphical representation of one embodiment of an ultrasound image with an overlaid grid;

FIG. 4 is a graphical representation of another embodiment of an ultrasound image with an overlaid grid transformed as a function of local estimates of motion; and

FIG. 5 is a graphical representation of two different embodiments of detected breathing cycles.

### DETAILED DESCRIPTION OF THE DRAWINGS AND PRESENTLY PREFERRED EMBODIMENTS

A cycle, such as a breathing cycle or cardiac, is detected from ultrasound data. Other cycles may be detected. In one embodiment, a long sequence of ultrasonic images is used to detect a curve indicating a breathing or other cycle. In a manner the same, similar or different than U.S. Pat. No. 6,659,953, the disclosure of which is incorporated herein by reference, motion is tracked throughout the sequence of images. The motion information is used to identify at least a portion of a cycle. The cycle information may be used to select trigger events or identify specific portions of the cycle. The cycle or specifically identified portions of the cycle are used for triggering, displaying a breathing cycle or other cycle waveform, or altering other processes. For example, the morphing processes disclosed in U.S. Pat. No. 6,659,953 are altered by resetting a reference frame to a different frame of data for each particular breathing cycle.

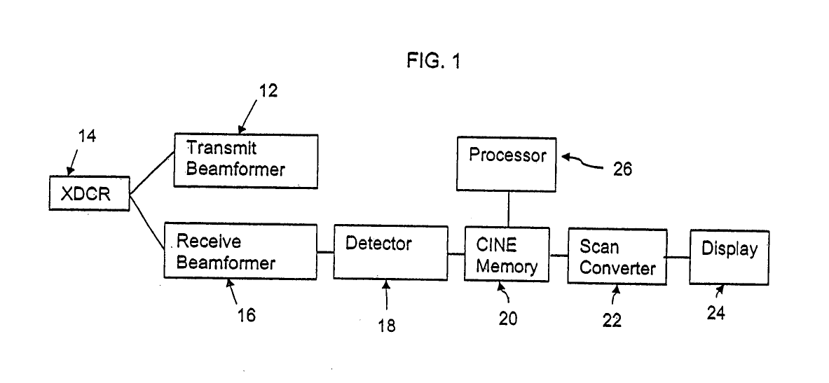

FIG. 1 shows one embodiment of a system 10 for detecting breathing or other cycle information with ultrasound data. The system 10 includes a transmit beamformer 12, a transducer 14, a receive beamformer 16, a detector or detectors 18, a CINE memory 20, a scan converter 22, a display 24 and a processor 26. Additional, different or fewer components may be provided. For example, ultrasound images are acquired in any of various now known or later developed processes and provided to the memory 20. The memory 20, processor 26 and display 24 comprise as a workstation for detecting the cycle information or other purposes with or without any of the other components of the system 10. As another example, the memory 20 is positioned after the scan converter 22 or before the detector 18.

The transmit beamformer 12 generates a plurality of electrical signals or a plane wave signal. In response, the transducer 14 generates acoustic energy focused along one or more scan lines or as a plane wave in any given transmit event. Acoustic echo signals impinge on the transducer 14. The transducer 14 generates electrical signals at each of a plurality of elements in response to the echoes. In response to the received electrical signals, the receive beamformer 16 generates data representing one or more scan lines. By repeating the transmit and receive sequences, a region of a patient is scanned, such as in a linear, sector; Vectorg, curved linear or other scanned format. One, two or three-dimensional scans may be used for acquiring each frame of data.

By repetitively scanning the region, a sequence of images representing a same region is obtained. The transducer 14 is held in one position to repetitively scan a substantially same region. In one embodiment, substantially no movement of the transducer is provided. Since users may unintentionally move the transducer 14 during imaging, some movement of the transducer 14 relative to the region may occur. As used herein, a substantially stationary or substantially same region is used to account for unintentional movement of the transducer 14. The acquired images are assumed to have no motion due to transducer 14 movement. Alternatively, any intentional or unintentional motion is detected and used to spatially align a series of images.

The detector 18 comprises one or more of a B-mode detector, a contrast agent detector, a Doppler detector, a color flow detector, or other detectors now known or later developed to detect a characteristic of received signals. In one embodiment, the detector 18 comprises both a B-mode detector and a contrast agent detector. The B-mode detector detects intensity or an envelope magnitude or amplitude of the received signals at each spatial location within the scanned region. The contrast agent detector comprises a B-mode detector optimized to detect contrast agent as opposed to tissue and fluid, such as a B-mode detector with a filter and/or scan sequence for detecting intensities at a second harmonic or other harmonic of the fundamental transmitted frequency. In another embodiment, the contrast agent detector detects a magnitude or amplitude of a difference or loss of correlation between two or more sequentially transmitted pulses to a same or adjacent spatial locations. Since contrast agents move or are destroyed by acoustic energy, a first pulse includes response from the contrast agent and a second pulse includes a lesser, different or no response from a destroyed or moved contrast agent. The difference between the two pulses isolates contrast agent information from stationary tissue information. In this embodiment, the contrast agent detector is a Doppler detector with a clutter filter adapted for loss of correlation detection, but other contrast agent detectors may be used. Other now known or later developed contrast agent detection techniques may be used. In alternative embodiments, the tissue region is free of added contrast agents throughout an entire imaging session.

The CINE memory 20 comprises a RAM, tape, hard drive, optical storage, or other device for storing a sequence of ultrasound images. In alternative embodiments, the memory 20 is configured using other formats than a CINE format, such as a computer memory or other memory for storing JPEG, MPEG, DICOM or other image information. The memory 20 is operable to store frames of ultrasound data acquired over a period of time. Each image represents a substantially same scanned region, such as a scanned region associated with substantially no transducer movement. As used herein, image or frame of data includes data at any point within the processing path, such as at the output of the receive beamformer, detectors 18, scan converter 22, or data as actually displayed as an image on the display 24. Each image within the stored sequence of images represents the region at different times.

The scan converter 22 comprises one or more processors or filters for converting data in a polar coordinate format as output by the receive beamformer 16 into a Cartesian coordinate format for the display 24. In alternative embodiments, the scan pattern is associated with a Cartesian coordinate format and the scan converter is optional. The scan converter 22 interpolates input data from one format into output data on a different format, such as interpolating the pixel information from data representing one, two or more adjacent spatial locations in a different format. In other embodiments, the scan converter 22 is a three-dimensional image processor for rendering three-dimensional representations.

The display 24 comprises a CRT, LCD, flat screen, plasma screen, an LED display, printer, charting device, or other devices for generating an image or a curve as a function of time. The display 24 displays the images in sequence for subjective assessment of perfusion or other diagnosis by a user. Alternatively or additionally, the display 24 generates a curve representing intensity or other image characteristic at one or more spatial locations as a function of time. For example, the display 24 displays a breathing or other cycle waveform. Other calculated parameters at a given time or over a range of times may be calculated and displayed by the display 24.

The processor 26 comprises one or more general processors, digital signal processors, application specific integrated circuits, analog devices, digital devices and combinations thereof. In one embodiment, the processor 26 is a personal computer, motherboard, personal computer processor, and/or personal computer video card or video processor. Through a bus or other electrical connection, the processor 26 receives the images from the CINE memory 20. In alternative embodiments, the processor 26 connects with the output of the scan converter or other component within the system 10 for receiving images. In yet other alternative embodiments, the processor 26 is included along the data path between the receive beamformer 16 and the display 24. The processor 26 operates in real-time or off-line.

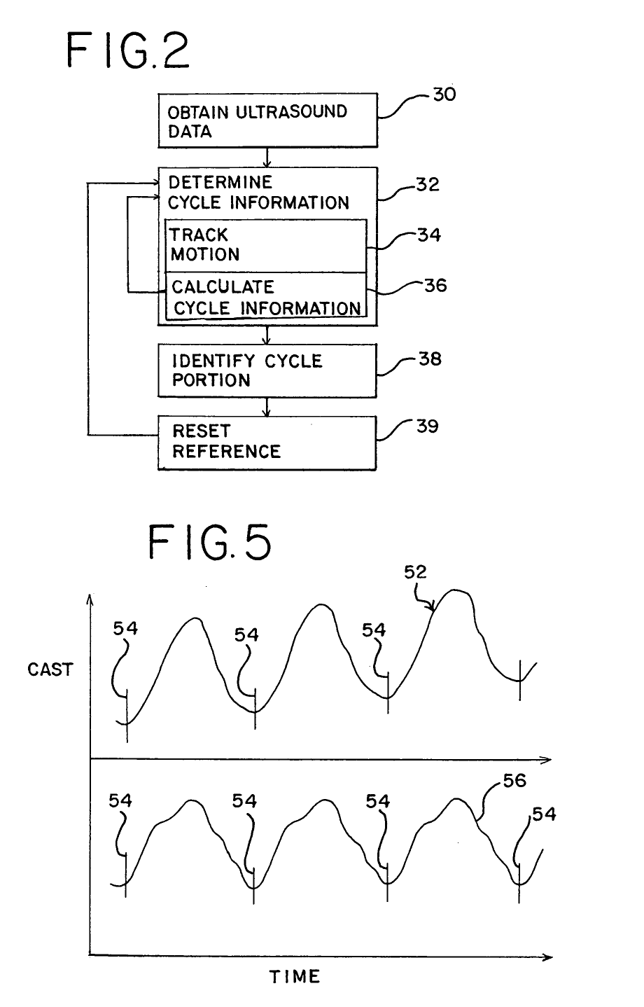

The processor 26 is operable to determine at least a first portion of a breathing or other cycle as a function of ultrasound data. For example, the processor 26 implements the local motion estimation and image transforms discussed below for FIG. 2. The processor 26 determines a motion parameter for a sequence of frames of ultrasound data relative to a reference frame of data. In one embodiment, motion is estimated at each of a plurality of local locations within an image. The motion is estimated between two different ultrasound images, the reference frame and another frame of data. The motion data is used to determine the cycle, such as the processor 26 calculating a cost as a function of the local estimates of motion. Other estimates may be used to calculate the cost.

The processor 26 may additionally use the identified cycle information for other processes. For example, the processor 26 identifies a specific portion of the breathing cycle as a function of a trend in the breathing cycle. The beginning of inhalation, exhalation or another specific portion of the breathing cycle is identified. Alternatively, an identifiable or repeating specific portion of the breathing cycle is identified without relationship to inhaling or exhaling.

The processor 26 implements other functions in other embodiments, such as implementing graphic user interface or control functions. In one example, the processor 26 filters or provides spatial smoothing of the estimated motions or calculated cycles. Low pass spatial filtering avoids overly drastic estimates of motion. The filter characteristics are determined as a function of the application or expected amount of motion.

FIG. 2 is a flowchart showing one embodiment of a method for detecting a cycle with ultrasound data. For example, breathing cycle information is detected with ultrasound. Different, additional or fewer acts may be provided in the same or different order than shown in FIG. 2. For example, acts 30, 32 and 36 are provided without acts 38 and 39. As another example, cycle information is calculated without tracking the motion in act 34.

In act 30, ultrasound data acquired over a period of time is obtained. The ultrasound data is obtained in real time with acquisition of each frame of data in one embodiment. In other embodiments, a sequence or clip of ultrasound data acquired over a period of time is obtained at a later given time. The obtained ultrasound data includes a sequence of ultrasound images or frames of ultrasound data. As used herein, an image may include frames of data that have not been used to generate a display or frames of data not yet formatted for display. Frames of data may include displayed or undisplayed data.

The sequence of images includes at least two ultrasound images representing a substantially same region without transducer movement or responsive to a substantially stationary transducer position. The region may include tissue and fluid structures that may move due to breathing, unintentional transducer movement, cyclical motion caused by the cardiac cycle, or other undesired sources of movement of the imaged tissue or the transducer 14 relative to the tissue. Alternatively, one or more images may include some transducer movement. Any number of ultrasound images may be included in the sequence, such as 300 or more images. One of the images within the sequence or an image not within the sequence is selected as a reference image. In one embodiment, the reference image corresponds to one of the extremities of inhalation or exhalation. For example, a first image within the sequence is automatically selected by the system 10. In other embodiments, other images, such as the last image, an image in the middle of the sequence or an image corresponding to a detected specific phase of a cycle, are automatically or manually selected by the system 10 or the user, respectively as a reference image. The reference image represents a common spatial frame of reference for motion tracking of other images to the same spatial frame of reference.

In one embodiment, each of the images within the sequence of images include one type of data, such as B-mode data, Doppler data, color flow data, contrast agent data or another type of data. In other embodiments, one or more of the images, such as all of the images, include two or more types of data, such as B-mode data and contrast agent data. The different types of data are either combined and provided as a single value or are separate. For example, contrast agent and B-mode data are provided as separate sets of values. Each set of values corresponds to a same or different portion of the imaged region, such as each type of data corresponding to exclusive spatial portions of the imaged region. For any given spatial location, either a contrast agent or a B-mode value is provided. In other embodiments, a B-mode value and a contrast agent value are both provided for a same spatial location. Each image represents any of various portions of the body, such as the liver, thyroid or breast.

In act 32, at least a first portion of a breathing or other cycle is determined as a function of ultrasound data. The determination of cycle information is performed using the act 34 of tracking motion and act 36 of calculating the cycle information from the tracked motion. Cycle information includes a waveform containing at least a part of a cycle, a value derived from a waveform, or one or more samples of the cycle waveform. Other processes for determining cycle information may be used, including now known or later developed processes.

In act 34, a motion parameter is determined as a function of a current frame of data relative to a reference frame of data. Current frame of data is used to indicate a selected frame of data as opposed to or as well as a most recently acquired frame of data. Motion is tracked for each of a plurality of frames of ultrasound data with respect to the reference frame of ultrasound data. The cycle information is determined as a function of the reference frame of data and one or more other frames of ultrasound data, such as subsequent frames of ultrasound data.

All of the images or a subset of the images in the sequence of images are tracked relative to the same reference image. For a sequence of three or more images, motion at a plurality of locations within each image of the sequence is estimated relative to a same reference image.

A global motion or a single local motion is tracked. In another embodiment, the mapping is performed as a function of local estimates of motion. Tissue in different portions of the imaged region may move by different amounts in response to any of the sources of movement discussed herein. Motion is estimated in different local locations of one image relative to the reference image to account for the differences in movement throughout the entire image. Spatial locations within any of the images are tracked to a substantially same tissue location throughout the images. The motion at each of a plurality of local locations is estimated. Any of various processes now known or later developed for estimating local motion are used, such as optical flow as discussed in U.S. Pat. No. 5,503,153, the disclosure of which is incorporated herein by reference.

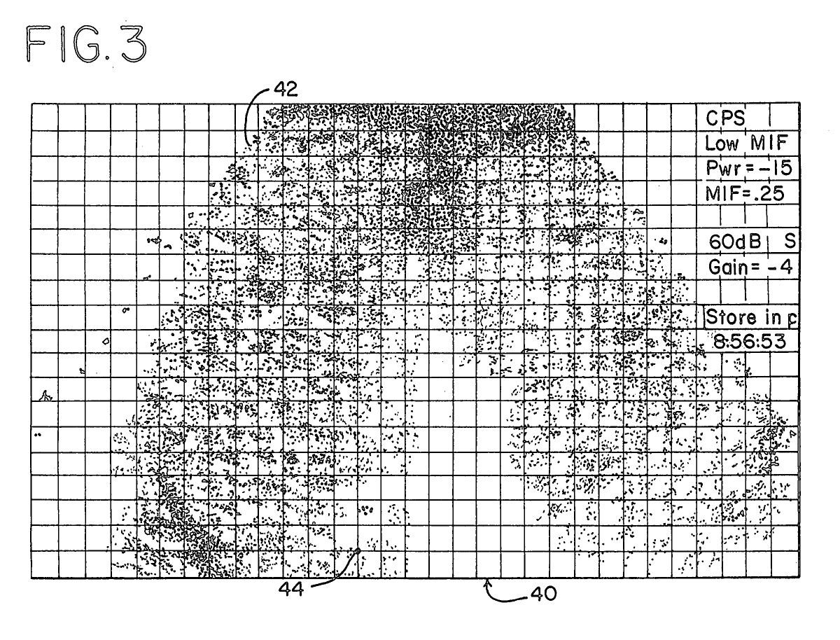

In one embodiment, a block matching motion analysis is used. Data representing a plurality of local areas of an image is separately correlated with data in the reference image. In one embodiment, the local estimates of motion correspond to an overlaid grid. FIG. 3 shows a grid 40 overlaid on a sector image 42. The grid 40 comprises a rectangular grid, but other grids may be used, such as triangular, polar, sector, vector, curved-vector, curvilinear, hexagonal or arbitrary grids. As shown, a grid point 44 or intersection of two grid lines is provided every 16th pixel. The grid 40 establishes a plurality of 16 pixel by 16 pixel regions. Other sized grids may be used, such as providing a grid line at every 8 pixels. The grid line spacing may vary. Each intersection of the grid lines or a grid point 44 defines a location for estimating motion. For each grid point 44, data representing an area around the grid point is correlated with data from another image, such as the reference image. The area around the grid point 44 corresponds to a same size as the grid spacing, such as a 16 by 16 pixel area surrounding the grid point, but other spacings larger than or smaller than the grid sampling size may be used.

Cross-correlations, correlation by minimizing the sum of absolute differences, maximizing the product, or other methods for correlating one data set with another data set may be used. The data for the area around each grid point 44 is compared to the data around a similar spatial location in the reference image. The data is then translated left and right and up and down in one pixel increments to identify the best correlation. In an alternate or additional embodiment, the data may also be rotated to identify the best correlation. The translation of the data extends along a 16 pixel range in both dimensions such that the center of the search area data is positioned at every pixel within a 16 by 16 pixel area on the reference image. Other search patterns using adaptive searching, skipping pixels, a greater or lesser range of searching or other differences may be used. For example, where the effects of the undesired motion are likely in one direction, a search pattern may be refined to search first or primarily along a direction of expected motion. A correlation threshold may indicate a proper correlation along an expected path of motion. In addition to correlation by shifting the data around each grid point using left-right and up-down directions, correlation may also be done by rotating the data around each grid point. In alternative embodiments, the motion is estimated as a function of Doppler data and direction of movement information. Other techniques now known or later developed for estimating motion at different local locations of one image with respect to another image may be used. In one embodiment, Pentium MMX/SSE2 instructions are used for determining the correlations.

In one embodiment, the local estimates of motion are filtered. For example, a low pass spatial filter filters the estimated motion vectors or magnitude of the translation of each grid point 44 relative to other grid points 44. The estimates of motions are filtered by spatially averaging over a 3×3 grouping of adjacent local estimates of motion, but unequal weightings, different spatial distributions or other spatial filtering may be provided. In alternative embodiments, no filtering is provided. In yet other alternative embodiments, an analysis or thresholding is applied to identify estimates of motion that are likely erroneous. Any erroneous estimates of motion are discarded and replaced by a further estimate of motion or by interpolating an estimate of motion from adjacent estimates of motion. Temporally or spatially adjacent estimates may be used. Temporal filtering may be used where estimated local motions are expected to vary similarly as a function of time. The estimated local motions are filtered prior to further warping of the image.

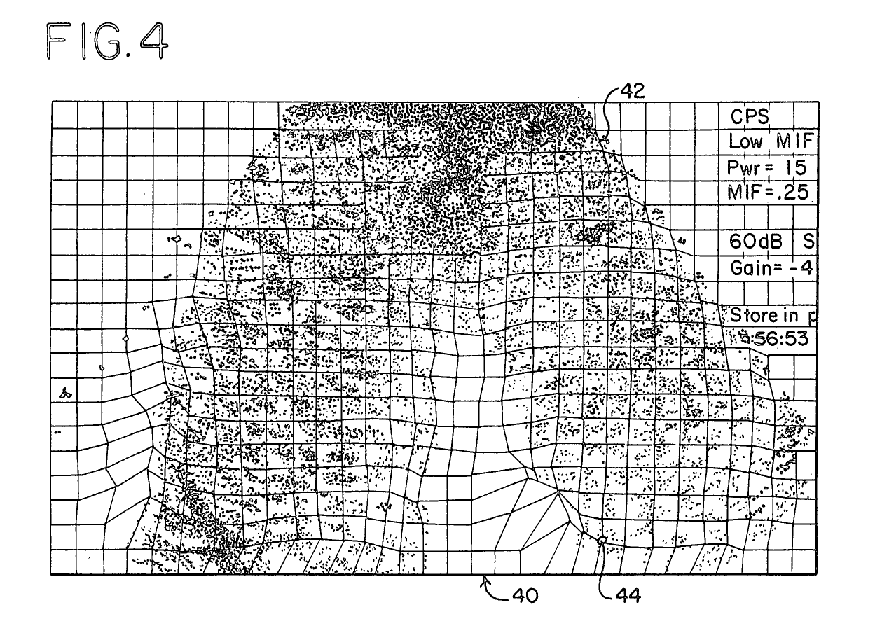

The grid points 44 are conceptually shifted as a function of the estimates of motion as shown in FIG. 4. The estimate of motion provides a motion vector or a magnitude and direction of motion corresponding to the grid point 44 within an image relative to the reference image. As shown in FIG. 4, different grid points are shifted in different directions and by different amounts, resulting in a grid 40 with a plurality of different sizes and shapes of quadrilaterals. In alternative embodiments, the shifts are limited to shifts along a single axis. Grid points 44 along the edge of the image 42 may be held stationary or shifted as a function of a changing amount of shift of adjacent but more interior grid points 44. Where the correlation is provided more inwardly within the image for an edge point, the edge grid points are shifted based on the correlation instead.

In act 36, a cyclic parameter is calculated as a function of the tracked motion. Any of various cyclic parameters may be calculated, such as a waveform corresponding to the cycle. In one embodiment, a cost function value is determined as a function of time. The cost function is associated with motion between the different frames of data. For example, a cost is calculated as a function of an amount of motion of each of a plurality of frames of ultrasound data relative to a reference frame of data. Using the local motion estimates discussed above, the grid points in the reference frame of data are mathematically represented as (xᵢⱼ⁰, yᵢⱼ⁰) where i and j identify the grid point 44. The corresponding grid points 44 in a different frame of data, k, after transforming the grid points into the reference frame, are mathematically represented by (xᵢⱼ^{k},yᵢⱼ^{k}) Using these mathematical representations, one exemplarily cost function is:

$${C}_{k} = {\sum }_{i = 0}^{N - 1} {\sum }_{j = 0}^{M - 1} \sqrt{{( {x}_{ij}^{k} - {x}_{ij}^{0} )}^{2} + {( {y}_{ij}^{k} - {y}_{ij}^{0} )}^{2}}$$

Since the cost function uses squared sums, an absolute value results. The cost function identifies a rectified cycle waveform. In one embodiment, a rectified or other non-absolute cycle waveform is sufficient. To obtain an absolute breathing or other waveform, a reference frame is selected as a frame likely associated with one of the extremes of inhalation or exhalation. This can be accomplished by first starting with the rectified waveform and then selecting the phase associated with the nearest peak of the rectified waveform. Once the nearest peak is found, the peak corresponds to either the extreme exhalation or extreme inhalation. The frame corresponding to this peak can be used as the reference frame. The cost function generated by using this reference frames yields the non-rectified breathing waveform. The cost as a function of time identifies at least a portion of a cycle waveform. If cost is calculated over a sufficient time, the cost may correspond to one or more cycles, such as one or more breathing cycles over a few second time period. Other forms of cost functions, such as sum of absolute differences in x and y or sum of differences in x and y can also be used.

FIG. 5 shows the cost plotted as a function of time or frame number, k, providing the breathing cycle waveform 52. Since the reference image is associated with a low cost value, the breathing cycle waveform 52 is similar to or corresponds to an absolute breathing waveform. The periodic nature of the cost shows the breathing cycle. The breathing cycle waveform 52 is displayed in one embodiment, but may be used without display for triggering or other processes in other embodiments. As shown in FIG. 5, the breathing cycle waveform 52 tends to diverge from the baseline cost or has a generally upward cost slope due to the accumulation of tracking errors. In alternative embodiments, no tracking errors are provided, resulting in a non-diverging waveform. The cost information or determined portion of the breathing cycle is used even with tracking errors. The calculation of cycle information continues or cycles back to the determination of cycle information in act 32 for determining the cost or other function as a function of time.

In an alternative embodiment, the divergence of the cost from a baseline is minimized or eliminated by tracking motion for each of the breathing cycles as a function of a reference frame specific to that cycle. By resetting the reference frame, accumulation of tracking errors may be avoided or minimized.

In act 38, a specific portion of a cycle is identified from the cycle information for assigning or resetting the reference frame. A cyclic parameter, such as the cost, is used to identify the specific location, such as the minimum marked by the markers 54 for each cycle. In one embodiment, local minimums are identified using thresholding or other comparative processes. In one embodiment, a five-sample or other number of sample sliding window is shifted as a function of time along with the cost values. The derivate of the cost value at each of the five points is computed. If the derivates all have positive signs, the cost function is increasing. If the derivates all have negative signs, the cost function is decreasing. The sign of the majority of the five values is computed. The majority can be 4 out of 5. Other numbers can be used. If the sign of the majority of the five values change from positive to negative as the window moves along the time direction, a maximum of the waveform is identified. If the sign of the majority of the five values changes from negative to positive, a minimum of the waveform is identified. Other processed using a window, comparison of samples, averaging, subtraction, other mathematical functions or other now known or later developed processes may be used for identifying a specific location within a cyclical pattern.

In act 39, the reference frame of data is reset. The motion tracking, calculation of the cyclic parameter and identification of a portion of a cycle is repeated for each of a plurality of subsequent cycles. For each repetition, the cycle or cyclic parameter is determined with a different reference frame. The reference frame of data to be used for each subsequent cycle is reset as a frame of ultrasound data corresponding to the identified portion of the cycle. For example, a frame of data corresponding to each minimum 54 is used as the reference frame of data for a given cycle. By identifying the reoccurrence of the same portion of the breathing cycle or other cycle, such as the reoccurrence of the minimum 54, the determination of the cycle information is repeated with different frames of reference associated with the reoccurrence of each cycle. In alternative embodiments, the reference frame of data is reset more frequently, such as at different phases within a cycle, or less frequently, such as every two or more cycles. Tracking may also be performed forward or backward in time. Forward and backward tracking results may be combined to improve the accuracy of the breathing waveform or its robustness.

In one embodiment, each reference frame is normalized to a same cost, such as by identifying an adjustment factor between the current reference frame and an original reference frame. The adjustment factor is then applied to all subsequent frames within the cycle for the given reference frame. Using the cost function of above, the shift automatically occurs as the cost is calculated as a difference from the reference frame and each reference frame will differ from itself by zero.

The divergence of the cost from a baseline is minimized or eliminated by tracking motion for each of the breathing cycles as a function of a reference frame specific to that cycle. Starting from the very first reference frame, n(0), the cost function is computed as before. The frame corresponding to the next minimum of the cost function, n(1), is detected by using the technique described above. The frame n(1) then becomes the reference frame for the next cycle. All frames in the next cycle are then transformed to n(1) and the cost function is computed accordingly. The above process is repeated for all the frames in the clip. By resetting the reference frame, accumulation of tracking errors may be avoided or minimized. Identifying the nearest preceding reference frame as, n(k), the cost function is now computed as ((\hat{X}^{k}ᵢⱼ, ^{k}ᵢⱼ), corresponding to the coordinates of the k^{th} frame transformed to the nearest preceding reference frame n(k).)

$${S}_{k} = {\sum }_{i = 0}^{N - 1} {\sum }_{j = 0}^{M - 1} \sqrt{{( {\hat{x}}_{ij}^{k} - {\hat{x}}_{ij}^{n ( k )} )}^{2} + {( {\hat{y}}_{ij}^{k} - {y}_{ij}^{n ( k )} )}^{2}} .$$

By repeating the determination of cycle information in act 32 after each resetting of the reference frame in act 39, a breathing cycle or cycle waveform 56 with minimal or no divergence results. As shown in FIG. 5, at each of the identified minimum locations 54, the cost function is reset to a same cost level. Within each cycle, the cost is calculated relative to the nearest preceding reference frame, such as the frame of ultrasound data corresponding to the first minimum 54 portion of each cycle.

Using either of the corrected or uncorrected cycle waveforms 52, 56 or a portion of a cycle waveform corresponding to less than one cycle, one cycle or more than one cycle, triggering or other processes may be performed without a separate breathing sensor. For diagnostic purposes, different phases of the respiratory cycle or other cycle, such as inhalation and exhalation, may be detected by locating the peaks and valleys of the waveform. A reoccurring portion of the waveform may be used for triggering imaging, injection of contrast agents, or other processes. Other now known or later developed respiratory gating of two- or three-dimensional imaging may be provided based on the identified breathing cycle.

In an alternative or additional embodiment, the breathing cycle waveform is displayed. For example, at least a portion of the waveform is displayed for viewing and associated diagnosis by the user.

In yet another alternative or additional embodiment, frames of ultrasound data are morphed within each cycle, such as each breathing cycle, to the reset or reassigned reference frame corresponding to the associated breathing cycle. New frames of data are morphed for each cycle relative to the reset frame of reference for the corresponding cycle. The morphing is performed as discussed in U.S. Pat. No. 6,659,953 or other now known or later developed forms of morphing. Accumulation of motion errors may be eliminated or minimized by resetting the reference frame for each cycle or other periodic resetting as discussed above.

In the morphing taught in U.S. Pat. No. 6,659,953, the grid 40 is warped based on the local estimates of motion. For each image of the sequence of images, the image is warped as a function of local motions estimated for the respective image. For example, the image 42 is warped as a function of the adjusted or warped grid 40 shown in FIG. 4. Warping the image data to correspond to the local estimates of motion results in images having suppressed motion relative to the reference image. To warp the image data based on the shifted grid 40, the image data is interpolated as a function of the local estimated motions or shifted grid 40. In one embodiment, the data within a grid box prior to warping is mapped or deformed into the quadrilateral after warping the grid 40. The data is linearly interpolated to evenly space the data based on the warped grid or estimates of motion. In one embodiment, texture mapping is provided using OpenGL commands to linearly interpolate the data in two dimensions. In other embodiments, other graphical application programming interfaces, such as DirectX, Direct3D or GDI+, all by Microsoft Corporation, Redmond, Wash., or interfaces by others may be used. Nonlinear interpolation may be used. Interpolation warps the data for any given area of the image defined by the original grid 40 into the area defined by the quadrilaterals of the adjusted or shifted grid 40 of FIG. 4. The entire image is warped as a function of a plurality of separate warping interpolations performed for separate local locations or areas. Local warping results in an image with spatial locations representing the same tissue as same spatial locations of the reference image. In an alternative embodiment, the data is translated without warping, and any spatial locations or pixels corresponding to an absence of data due to a difference in local estimated motion are filled by interpolation or extrapolation.

As an alternative or an additional act, a time-intensity curve is determined in addition to the cycle information. In one embodiment, the time-intensity curve is determined using contrast agent data or data representing a spatial location associated with the perfusion of contrast agent. Other types of data may be used for any of various time-intensity or other calculations. A change as a function of time for a same spatial and associated tissue location is determined from two or more images within the sequence after warping. Since the images are warped to a common reference image, a same spatial location in each image represents a same tissue location. For quantities associated with more than one cycle or associated with a reset reference frame, the position of the reset reference frame relative to the original reference frame may be used to adjust the morphing during later cycles. The adjustment avoids accumulated motion tracking errors by reducing the number of frames for which tracking is done but more likely maintains the spatial reference over multiple cycles. Each spatial location within each image in the sequence represents a substantially same tissue, resulting in more accurate parametric images and calculations of perfusion. In one embodiment, abnormal image intensity modulations due to the stretching or compression of warping are compensated by modulating the image intensities with the Jacobian of the transformation or other filtering.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for detecting breathing cycle information with ultrasound, the method comprising:
(a) obtaining ultrasound data acquired over a period of time; and
(b) determining at least a first portion of a breathing cycle as a function of the ultrasound data.

2. The method of claim 1 wherein (a) comprises obtaining ultrasound data responsive to contrast agents.

3. The method of claim 1 wherein (b) comprises determining a motion parameter of a current frame of data relative to a reference frame of data.

4. The method of claim 4 wherein (b) comprises determining the motion parameter as a function of a plurality of local regions in the current frame of data relative to the reference frame of data.

5. The method of claim 1 wherein (b) comprises determining a cost function value as a function of time, the cost function value associated with motion between a plurality of frames of data.

6. The method of claim 1 further comprising:
(c) displaying a breathing cycle waveform comprising the first portion.

7. The method of claim 1 further comprising:
(c) identifying the first portion as a function of a trend in the breathing cycle.

8. The method of claim 7 wherein (c) comprises identifying one of a peak and minimum of the breathing cycle.

9. The method of claim 1 wherein (b) comprises determining the first portion as a function of a first reference frame of ultrasound data and a first subsequent frame of ultrasound data;
further comprising:
(c) identifying reoccurrence of the first portion of the breathing cycle; and
(d) repeating (b) with a second reference frame of ultrasound data associated with the reoccurrence of the first portion.

10. The method of claim 1 further comprising:
(c) repeating (b) for each cycle of the breathing cycle with a different reference frame for each breathing cycle; and
wherein (b) comprises tracking motion for each breathing cycle as a function of the reference frame for each breathing cycle.

11. The method of claim 10 further comprising:
(d) morphing frames of ultrasound data within each breathing cycle to the reference frame for the corresponding breathing cycle.

12. A system for detecting breathing cycle information with ultrasound, the system comprising:
a memory operable to store frames of ultrasound data acquired over a period of time; and
a processor operable to determine at least a first portion of a breathing cycle as a function of the ultrasound data.

13. The system of claim 12 wherein the processor is operable to determine a motion parameter of a plurality of frames of ultrasound data relative to a reference frame of data.

14. The system of claim 12 further comprising:
a display operable to display a breathing cycle waveform.

15. The system of claim 12 wherein the processor is operable to identify the first portion as a function of a trend in the breathing cycle.

16. A method for detecting a cycle with ultrasound data, the method comprising:
(a) tracking motion of a plurality of frames of ultrasound data with respect to a reference frame of ultrasound data;
(b) calculating a cyclic parameter as a function of the tracked motion;
(c) identifying a first portion of the cycle as a function of the cyclic parameter;
(d) repeating (a), (b) and (c) for each of a plurality of subsequent cycles; and
(e) resetting the reference frame of data for each of the plurality of subsequent cycles as a first frame of ultrasound data corresponding to the first portion of the cycle.

17. The method of claim 16 wherein (a) comprises tracking the motion as a function of a plurality of local regions.

18. The method of claim 16 wherein (b) comprises calculating a cost as a function of an amount of motion of each of the plurality of frames of ultrasound data relative to the reference frame of data.

19. The method of claim 16 further comprising:
(f) morphing frames of data for each cycle relative to the reset reference frame for the corresponding cycle.

20. The method of claim 16 wherein (c) comprises identifying the first portion in a breathing cycle.

21. The method of claim 16 wherein (a) comprises tracking motion in B-mode frames of data.
